(19) United States
(12) Patent Application Publication
(10) Pub. No.: US 2010/0303777 A1
(43) Pub. Date: Dec. 2, 2010

(54) DELIVERY OF BINDING MOLECULES TO INDUCE IMMUNOMODULATION

(75) Inventors: An De Creus, Gavere (BE); Pieter Rottiers, De Pinte (BE)

Correspondence Address: KNOBBE MARTENS OLSON & BEAR LLP, 2040 MAIN STREET, FOURTEENTH FLOOR, IRVINE, CA, 92614, US

(73) Assignee: ActoGeniX N.V., Zwijnaarde (BE)

(21) Appl. No.: 12/518,843

(22) PCT Filed: Dec. 12, 2007
(86) PCT No.: PCT/EP07/63851

§ 371 Date: Jun. 11, 2009

(30) Foreign Application Priority Data
Dec. 14, 2006 (EP) 06025933.0
Apr. 19, 2007 (EP) 07106543.7
Jun. 4, 2007 (EP) 07109542.6

(51) Int. Cl. A61K 35/66; A61K 35/74; A61K 36/06; A61K 36/064; A61P 37/08; A61P 29/00; A61P 37/00; A61P 43/00; C12N 1/00; C12N 1/20; C12N 1/12

(52) U.S. Cl. 424/93.45; 424/93.1; 424/93.4; 424/93.51; 435/243; 435/252.1; 435/255.1; 435/252.9; 435/255.2

(57) ABSTRACT

Delivery of binding molecules, such as antibodies, antibody fragments, single antibody variable domains, soluble receptors, ligands and dominant negative variants, to induce an immunomodulation in a patient is disclosed. More specifically, a medicament which includes micro-organisms which produce the binding molecules is described for the treatment of immune mediated diseases.

The present invention relates to the delivery of binding molecules, such as antibodies, antibody fragments, single antibody variable domains, soluble receptors, ligands and dominant negative variants, to induce an immunomodulation in a patient. More specifically, the invention relates to the production of a medicament comprising said binding molecules producing micro-organisms, and the use of this medicament in the treatment of immune mediated diseases, preferably T-cell mediated diseases.

Immune mediated diseases are conditions which result from abnormal activity of the body's immune system (innate and adaptive). These diseases include allergies, auto-immune disorders, food intolerance, graft rejection, irritable bowel syndrome (IBS) and inflammatory diseases.

T-cell mediated diseases refers to inflammatory diseases, auto-immune diseases, organ and bone marrow transplant rejection and other disorders associated with T cell mediated immune response, including acute or chronic inflammation, allergies, contact dermatitis, psoriasis, rheumatoid arthritis, multiple sclerosis, type I diabetes, inflammatory bowel disease (IBD) Crohn's disease, ulcerative colitis, celiac disease, Guillain-Barre syndrome, graft versus host disease (and other forms of organ or bone marrow transplant rejection) and lupus erythematosus.

Inflammatory bowel disease (IBD) refers to a group of gastrointestinal or alimentary tract disorders characterized by a chronic non-specific inflammation of portions of the gastrointestinal tract. The most prominent examples of IBD in humans are ulcerative colitis (UC) and Crohn's disease (CD). The etiology or etiologies of IBD are unclear. IBD diseases appear to result from the unrestrained activation of an inflammatory response in the intestine. This inflammatory cascade is thought to be perpetuated through actions of proinflammatory cytokines and selective activation of lymphocyte subsets. UC and CD are associated with many symptoms and complications, including growth retardation in children, rectal prolaps, blood in stools, wasting, iron deficiency and anemia. UC refers to a chronic, non-specific, inflammatory and ulcerative disease having manifestations primarily in the colonic mucosa. It is frequently characterized by bloody diarrhea, abdominal cramps, blood and mucus in the stool, malaise, fever, anemia, anorexia, weight loss, leukocytosis, hypoalbuminemia and an elevated erythrocyte sedimentation rate.

The most commonly used medication to treat immune mediated diseases, such as T cell mediated diseases, includes anti-inflammatory drugs such as, for instance, corticosteroids and sulicilates, e.g. sulphasalazine and its derivatives. For patients that do not respond to these drugs, immunosuppressive drugs such as cyclosporine A, mercaptopurin and azathropine are used. However, these medicaments all have serious side effects.

A recent, successful development in the treatment of IBD and rheumatoid arthritis consists in the use of compounds, blocking the working of TNF or its receptor. In this respect, the use of TNF antibodies is one of the most promising new therapies. Tumor necrosis factor α (TNFα) is a cytokine produced by numerous cell types, including monocytes and macrophages, which was originally identified based on its capacity to induce the necrosis of certain mouse tumors (see e.g., Old, L. (1985) Science 230:630-632). TNFα has been implicated in the pathophysiology of a variety of other human diseases and disorders, including sepsis, infections, autoimmune diseases, transplant rejection and graft-versus-host disease (see e.g. Moeller, A. et al. (1990) Cytokine 2:162-169; U.S. Pat. No. 5,231,024 to Moeller et al; European Patent Publication No. 260 610 (B1) by Moeller, A. et al.; Vasilli. P. (1992) Annu. Rev. Immunol. 10:411-452; Tracey, K. J. and Cerami, A. (1994) Annu. Rev. Med. 45:491-503). Because of the harmful role of human TNFα (hTNFα) in a variety of human disorders, therapeutic strategies have been designed to inhibit or counteract hTNFα activity. In particular, antibodies that bind to, and neutralize, hTNFα have been sought as a means to inhibit hTNFα activity.

Several antibody preparations have been tested for the treatment of IBD. Although polyclonal antibodies have been tested in phase II clinical tests, monoclonal antibodies are clearly preferred. Infliximab is a chimeric human-mouse monoclonal antibody of the IgG1K subclass, which specifically targets and irreversibly binds to TNFα on cell membranes and in blood. Single intravenous doses, ranging from 5 to 20 mg/kg of the antibody infliximab resulted in a drastic clinical improvement in active Crohn's disease, it has been launched on the market to treat Crohn's disease in 1998.

To solve possible problems linked to chimeric antibodies, the human monoclonal TNFα adalimumab was developed, which is currently tested in phase III clinical trials for the treatment of Crohn's disease. To improve the half-life time of the antibody in patients, Celltech developed Certolizumab pegol, which is a humanized monoclonal pegylated anti-TNFα antibody, which is currently also tested in phase III clinical trials for the treatment of Crohn's disease.

However, in all those cases, the antibodies are applied in a systemic way, mainly by subcutaneous injection. Systemic administration of anti-TNF-α antibody may result in rather serious unwanted effects, including headache, abscess, upper respiratory tract infection and fatigue.

The unwanted effects associated with systemic delivery could be solved by local delivery on the place of the inflammation. A promising system for delivery of biological active compounds in the intestine has been disclosed in WO97/14806, whereby non-invasive gram positive bacteria such as lactic acid bacteria are used to deliver biological active compounds in the gut. WO00/23471 discloses that this system can be used to deliver IL-10 to the ileum, whereby this strain can be used to treat IBD. WO01/98461 discloses an alternative method for intestinal delivery using yeast. However, although the delivery of biologically active compounds is described, these documents do not teach the delivery of binding molecules, such as antibodies in the intestine. The in situ production of active binding molecules, such as antibodies in the intestine is far from straightforward, as both folding and secretion of these binding molecules, e.g. antibodies, are critical. Especially, the stabilization of the structure by sulfur bridges may cause problems for the production of antibodies in bacteria or yeasts. Moreover, whereas cytokines like IL-10 fulfil a catalytic function, antibodies or other binding molecules need to be produced in a sufficient amount to inactivate and/or neutralize the endogenous produced pro-inflammatory cytokines, chemokine and/or growth factor.

Surprisingly, we found that the local delivery of binding molecules, e.g. antibodies such as anti-TNFα antibody, anti-IL12p40 antibody, and an anti-IL23p19 antibody or dominant negative variants, such as a dnMCP-1 variant, by a genetically engineered micro-organism can be used in an efficient way to treat immune mediated diseases, such as IBD.

Throughout this disclosure, various publications, patents and published patent specifications are referenced by an identifying citation. The disclosures of these publications, patents and published patent specifications are hereby incorporated by reference into the present disclosure to describe more fully the state of the art to which this invention pertains.

The practice of the present invention will employ, unless otherwise indicated, conventional techniques of organic chemistry, pharmacology, molecular biology (including recombinant techniques), cell biology, biochemistry, and immunology, which are within the skill of the art. Such techniques are explained fully in the literature, such as, “Molecular Cloning: A Laboratory Manual” Second Edition (Sambrook et al., 1989); “Oligonucleotide Synthesis” (Gait, ed., 1984); “Animal Cell Culture” (Freshney, ed., 1987); the series “Methods in Enzymology” (Academic Press, Inc.); “Handbook of Experimental Immunology” (Weir & Blackwell, eds.); “Gene Transfer Vectors for Mammalian Cells” (Miller & Calos, eds., 1987); “Current Protocols in Molecular Biology” (Ausubel et al., eds., 1987, and periodicals) “Polymerase Chain Reaction” (Mullis et al., eds., 1994); and “Current Protocols in Immunology” (Coligan et al., eds., 1991).

As used herein, certain terms may have the following defined meanings. As used in the specification and claims, the singular form “a”, “an” and “the” include plural references unless the context clearly dictates otherwise. For example, the term “a cell” includes a plurality of cells, including mixtures thereof. Similarly, use of “a compound” for treatment or preparation of medicaments as described herein contemplates using one or more compounds of this invention for such treatment or preparation unless the context clearly dictates otherwise.

As used herein, the term “comprising” is intended to mean that the compositions and methods include the recited elements, but not excluding others per se. The term “comprising” comprises “consisting essentially of”. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention.

A first aspect of the invention is the use of a binding molecule producing micro-organism for the preparation of a medicament to treat immune mediated diseases, preferably T-cell mediated diseases. Preferably, the use of a genetically modified micro-organism, producing binding molecules, such as antibody, antibody fragment, dAb, bispecific antibody, trispecific antibody, multispecific antibody, bivalent antibody, trivalent antibody, multivalent antibody, VHH, nanobody, Fab, scFv, Fv, dAb, Fd, diabody, triabody, single chain antibody, single domain antibody, single antibody variable domain, soluble receptor, CTLD-derived binder, trimer-derived binder, ligand and/or dominant negative variants. The binding molecule is capable of binding to target molecules, such as proinflammatory cytokines or their receptors, chemokines, costimulatory molecules, adhesion molecules or enzymes resulting in the modulation of an inflammatory response in a patient.

The term “binding molecule”, as used herein, refers to a member of a pair of molecules which have binding specificity for one another, e.g. a binding molecule has a binding specificity for a target molecule. The members of a specific binding pair may be naturally derived or wholly or partially synthetically designed. One member of the pair of molecules has an area on its surface, or a cavity, which specifically binds to and is therefore complementary to a particular spatial and polar organisation of the other member of the pair of molecules. Thus the members of the pair have the property of binding specifically to each other. Examples of types of specific binding pairs are antigen-antibody, biotin-avidin, hormone-hormone receptor, ligand-ligand receptor, enzyme-substrate. Other examples of specific binding pairs include, carbohydrates and lectins, complementary nucleotide sequences (including probe and capture nucleic acid sequences used in DNA hybridization assays to detect a target nucleic acid sequence), complementary peptide sequences including those formed by recombinant methods, effector and receptor molecules, enzyme cofactors and enzymes, enzyme inhibitors and enzymes, and the like. Furthermore, specific binding pairs can include members that are analogues or fragments of the original specific binding molecule. In an embodiment, the binding molecule capable of binding a target molecule, such as a cytokine, provides a polypeptide according to the invention with a binding affinity (Kd) for the target molecule, e.g. a cytokine that preferably is 10⁻⁶ M, 10⁻⁷ M, 10⁻⁸ M or less determined by surface plasmon resonance. In other useful embodiments, the Kd value is less than 10⁻⁹ M, 10⁻¹⁰, 10⁻¹¹ M, 10⁻¹² M, 10⁻¹³ M, 10⁻¹⁴ M, or even less than 10⁻¹⁵ M. In further embodiments, the K-off rate for the trimeric polypeptide according to the invention is less than 1 as determined by plasmon resonance. The term “K-off”, as used herein, is intended to refer to the off rate constant for dissociation of a specific binding member from the specific binding molecule/cytokine complex. The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden). As mentioned above, it is preferred that the binding molecule according to the invention at least partially or fully blocks, inhibits, or neutralises a biological activity of a target molecule, such as a cytokine or chemokine. As used herein, the expression “neutralises” or “neutralisation” means the inhibition of or reduction in a biological activity of a cytokine as measured in vivo or in vitro, by methods known in the art, such as, for instance, as detailed in the examples. In particular, the inhibition or reduction may be measured by determining the colitic score or by determining the target molecule in a tissue or blood sample. As used herein, the expression “neutralises” or “neutralisation” means the inhibition of or reduction in a biological activity of a cytokine as measured in vivo or in vitro, by at least 10% or more, preferably by at least 20%, 30%, 40%, 50%, 60%, 70%, 80%, 90% and even more preferably by 100%.

Preferably, said binding molecules are binding to and inhibiting the biological effect of cytokines chosen from the list of IL-1β, IL-2, IL-4, IL-5, IL-6, IL-7, IL-8, IL-9, IL-12 (or its subunits IL-12p35 and IL12p40), IL-13, IL-15, IL-16, IL-17, IL-18, IL-21, IL-23 (or its subunit IL-23p19), IL-27, IL-32 (and its splice variants), IFN (α, β, γ) and TNFα. Preferably, said binding molecules are soluble cytokine receptors such as gp130, or are binding to the receptors of said cytokines, for example IL-2R (CD25, CD122, CD132), IL-12R (beta1, beta2), IL15R, IL-17R, IL-23R or IL6R, without triggering an inflammatory signal. Preferably, said binding molecules are neutralizing chemokines chosen from the list of MIF, MIP-1a, MCP-1, RANTES and Eotaxin. Preferably, said binding molecules are solving the blockade of immune activation via binding to costimulatory molecules from the list of CD3/CD28, HVEM, B7.1/B7.2, CD40/CD40L(CD154), ICOS/ICOSL, OX40/X40L, CD27/CD27L(CD70), CD30/CD30L(CD153) and 41BB/41BBL. Preferably, said binding molecules are solving the blockade of inflammation via binding to adhesion molecules from the list I-CAM1, α4 integrin and α4β7 integrin. Preferably, said binding molecules have a costimulatory and agonistic effect on CD3, CTLA4 and/or PD1. Preferably, said binding molecules are neutralizing T-cells or B-cell activity by targeting CD25, CD20, CD52, CD95, BAFF, APRIL and/or IgE. Preferably, said binding molecules are solving the blockade of inflammation via binding to enzymes from the MMP family. Preferably, said binding molecules assert an anti-angiogenic effect, such as neutralizing αvβ3/α5β1 and IL-8 activity. In a further preferred embodiment said binding molecule is capable of neutralizing the biological effect of TNFα, IL-12, IFNγ, IL-23 or IL-17. Preferably, said binding molecule is chosen from the group consisting of
an anti-TNFα antibody, anti-TNFα antibody fragment, anti-TNFα single antibody variable domain, soluble TNF receptor or dominant negative variant of TNFα;
anti-IL-12 antibody, anti-IL-12 antibody fragment, anti-IL-12 single antibody variable domain, soluble IL-12 receptor, dominant negative variant of IL-12 or IL-12 dAb;
anti-IL-12p35 antibody, anti-IL-12p35 antibody fragment, anti-IL-12p35 single antibody variable domain, soluble IL-12p35 receptor, dominant negative variant of IL-12p35 or IL-12p35 dAb;
anti-IL-12p40 antibody, anti-IL-12p40 antibody fragment, anti-IL-12p40 single antibody variable domain, soluble IL-12p40 receptor, dominant negative variant of IL-12p40 or IL-12p40 dAb;
anti-IL-23 antibody, anti-IL-23 antibody fragment, anti-IL-23 single antibody variable domain, soluble IL-23 receptor, dominant negative variant of IL-23 or IL-23 dAb;
anti-IL-23p19 antibody, anti-IL-23p19 antibody fragment, anti-IL-23p19 single antibody variable domain, soluble IL-23p19 receptor, dominant negative variant of IL-23p19 or IL-23p19 dAb;
an anti-IFNγ antibody, anti-IFNγ antibody fragment, anti-IFNγ single antibody variable domain, soluble IFNγ receptor or dominant negative variant of IFNγ;
anti-IL-17 antibody, anti-IL-17 antibody fragment, anti-IL-l7single antibody variable domain, soluble IL-17 receptor, dominant negative variant of IL-17 or IL-17 dAb; and
anti-MCP-1 antibody, anti-MCP-1 antibody fragment, anti-MCP-1 single antibody variable domain, soluble IL-17 receptor, dominant negative variant of MCP-1 or MCP-1 dAb.

The present invention relates also to a binding molecule having an antagonizing or agonistic activity of the target molecule. In this invention, the term “antagonist” or “antagonizing activity” refers to an interaction between chemicals in which one, i.e. the binding molecule, partially or completely inhibits or neutralises the effect of the other, i.e. the target molecule, in particular agents having high affinity for a given receptor, but which do not activate this receptor. In this invention, the term “agonist” or “agonistic activity” relates to an agent, i.e. the binding molecule, which both binds to a receptor and has an intrinsic effect.

Preferably, said genetically modified micro-organism is a lactic acid bacterium or a yeast. Delivery of biologically active polypeptides into the animal body by lactic acid bacteria has been disclosed in WO9714806; intestinal delivery of peptides by yeast has been described in WO0198461. However, none of these documents mention the delivery of binding molecules in the intestine. Production, secretion and delivery in vivo of biological active binding molecules is far from evident, as a correct folding and secretion of the binding molecule, such as for example antibody, antibody fragment, dAb, bispecific antibody, trispecific antibody, multispecific antibody, bivalent antibody, trivalent antibody, multivalent antibody, VHH, nanobody, Fab, scFv, Fv, dAb, Fd, diabody, triabody, single chain antibody, single domain antibody, single antibody variable domain, soluble receptor, CTLD-derived binder, trimer-derived binder, ligand and/or dominant negative variants is required, and sufficient secretion of such molecules is required to obtain a neutralizing activity.

Heterologous host cells, i.e. micro-organisms, for the production of recombinant proteins are known in the art, and can, for example, be a bacterium or yeast. Preferably, said micro-organism is a lactic acid bacterium. In one preferred embodiment said genetically modified micro-organism is a Lactococcus lactis strain, preferably said genetically a Lactococcus lactis ThyA mutant. A specially preferred embodiment is the use of a Lactococcus lactis ThyA mutant, wherein the gene encoding the binding molecule, e.g. an anti-TNF-α antibody, has been used to disrupt the THYA gene. In another preferred embodiment said lactic acid bacterium is a Lactobacillus sp.

In another preferred embodiment, yeast is be used to deliver the binding molecules. Preferably said yeast is a Saccharomyces sp, such as cerevisiae, even more preferably said yeast is Saccharomyces cerevisiae subsp. Boulardii. Active binding molecules of the invention, such as, for instance, CTLD-derived binders and trimer-derived binders, can easily be expressed in the micro-organisms of the invention with the benefit of significant reduced production costs and without limitations in production capacity.

IBD, as used here, includes but is not limited to chronic colitis, ulcerative colitis and Crohn's disease. Preferably, IBD is chronic colitis. The present invention thus provides the use as defined herein, wherein said immune mediated diseases are chosen from the group consisting of T-cell mediated diseases, inflammatory diseases, autoimmune and allergic diseases and organ and bone marrow transplant rejection. Preferably, said immune mediated disease is a T-cell mediated disease. In an alternative preferred embodiment, said T-cell mediated disease is Crohn's disease. In a further preferred embodiment, said T-cell mediated disease is ulcerative colitis.

Another aspect of the invention is a pharmaceutical composition for mucosal administration, comprising at least one genetically modified binding molecule producing micro-organism.

The binding molecule according to the invention can be any member of a pair of molecules having binding specificity for each other, as mentioned supra. Preferably, said binding molecule is an antibody, antibody fragment, dAb, bispecific antibody, trispecific antibody, multispecific antibody, bivalent antibody, trivalent antibody, multivalent antibody, VHH, nanobody, Fab, scFv, Fv, dAb, Fd, diabody, triabody, single chain antibody, single domain antibody, single antibody variable domain, soluble receptor, CTLD-derived binder, trimer-derived binder, ligand and/or dominant negative variants.

The binding molecule according to the invention can be produced by the micro-organism as a monomer or a multimer. The multimer may be an N-mer, wherein N≧2, e.g. a di-mer or a tri-mer. The multimer may be a homo-multimer, i.e. all moieties constituting the binding molecule are substantially identical, or the multimer may be hetero-multimer, i.e. not all of the moieties constituting the binding molecule are substantially identical. The person skilled in the art will appreciate that the present invention also relates to monomers which may polymerise to multimers.

When a molecule of the invention combines two (different of equal) functions, it is called bifunctional. Similarly, when a molecule of the invention combines three or more than three different or equal functions, it is called trifunctional, respectively multifunctional. When a molecule of the invention is combining two, three or more binding parts having a different specificity, it is called bi-, respectively tri- or multispecific. When a molecule of the invention is combining two, three or more binding parts having the same specificity, it is called bi-, respectively, tri- or multivalent for the binding specificity. Bivalent antibodies perform surprisingly better than monovalent antibodies. Although bivalent antibodies are larger than monovalent ones, it does not affect the production in Lactic acid bacteria, such as Lactococcus. It was found that the production of bivalent antibodies is at least as good if not better than for monovalent antibodies. The efficacy, e.g. neutralizing effect, of bivalent antibodies is more pronounced than that of monovalent antibodies.

In the present context, the term “antibody” is used to describe an immunoglobulin whether natural or partly or wholly engineered. As antibodies can be modified in a number of ways, the term “antibody” should be construed as covering any specific binding molecule or substance having a binding domain with the required binding specificity for the other member of the pair of molecules, i.e. the target molecule, as defined supra. Thus, this term covers antibody fragments, derivatives, functional equivalents and homologues of antibodies, as well as single chain antibodies, bifunctional antibodies, bivalent antibodies, VHH, nanobodies, Fab, scFv, Fv, dAb, Fd, diabodies, triabodies and camelid antibodies, including any polypeptide comprising an immunoglobulin binding domain, whether natural or wholly or partially engineered. Chimeric molecules comprising an immunoglobulin binding domain, or equivalent, fused to another polypeptide are therefore included. The term also covers any polypeptide or protein having a binding domain which is, or is homologous to, an antibody binding domain, e.g. antibody mimics. Examples of antibodies are the immunoglobulin isotypes and their isotypic subclasses, including IgG, IgA and IgE. The person in the art will thus appreciate that the present invention also relates to antibody fragments, comprising an antigen binding domain such as VHH, nanobodies Fab, scFv, Fv, dAb, Fd, diabodies and triabodies.

‘Dominant negative variants’ as used herein mean mutations that produce a protein that adversely affects the function of the normal, wild-type protein. The term ‘single antibody variable domain’ (dAb) as used herein refers to a folded polypeptide domain comprising sequences characteristic of antibody variable domains. It therefore includes complete antibody variable domains, single domain antibodies, such as VHH and nanobodies, and modified variable domains, for example in which one or more loops have been replaced by further sequences, or antibody variable domains which have been truncated or comprise N- or C-terminal extensions, as well as folded fragments of variable domains which retain at least in part the binding activity and specificity of the full-length domain. Moreover, the term dAb includes within its scope those single antibody variable domains in which one or more hypervariable loops and/or CDRs have been replaced with those from a second variable domain, which may be from the same or different origin.

The single domain antibodies may be joined to form any of the polypeptides disclosed herein comprising more than one single domain antibody using methods known in the art or any future method. For example, the single domain antibody may be fused genetically at the DNA level i.e. a polynucleotide construct formed which encodes the complete polypeptide construct comprising one or more anti-target single domain antibodies and one or more anti-serum protein single domain antibodies. A method for producing bivalent or multivalent single domain antibodies, i.e. VHH polypeptide constructs, is disclosed in PCT patent application WO 96/34103. One way of joining multiple single domain antibodies is via the genetic route by linking single domain antibody coding sequences either directly or via a peptide linker. For example, the C-terminal end of the first single domain antibody may be linked to the N-terminal end of the next single domain antibody. This linking mode can be extended in order to link additional single domain antibodies for the construction and production of tri-, tetra-, etc. functional constructs.

According to one aspect of the present invention, the single domain antibodies are linked to each other directly, without use of a linker. Contrary to joining bulky conventional antibodies where a linker sequence is needed to retain binding activity in the two subunits, polypeptides of the invention can be linked directly thereby avoiding potential problems of the linker sequence, such as antigenicity when administered to a human subject, instability of the linker sequence leading to dissociation of the subunits.

According to another aspect of the present invention, the single domain antibodies are linked to each other via a peptide linker sequence. Such linker sequence may be a naturally occurring sequence or a non-naturally occurring sequence. The linker sequence is preferably to be non-immunogenic in the subject to which the binding molecule is administered. The linker sequence may provide sufficient flexibility to the multivalent binding molecule, at the same time being resistant to proteolytic degradation. A non-limiting example of a linker sequences is one that can be derived from the hinge region of a single domain antibody, i.e. VHH, described in WO 96/34103.

VHHs, The variable domain derived from a heavy chain antibody naturally devoid of light chain is known herein as a VHH or nanobody as described in WO 94/04678, to distinguish it from the conventional VH of four chain immunoglobulins. Such a VHH molecule can be derived from antibodies raised in Camelidae species, for example in camel, dromedary, llama, alpaca and guanaco. Other species besides Camelidae may produce heavy chain antibodies naturally devoid of light chain; such VHHs are within the scope of the invention. VHH molecules are about 10 times smaller than IgG molecules. They are single polypeptides and very stable, resisting extreme pH and temperature conditions. Moreover, they are resistant to the action of proteases which is not the case for conventional antibodies.

Diabodies are a class of small bivalent and bispecific antibody fragments that can be expressed in bacteria and yeast in functional form and with high yields (up to 1 g/l). Diabodies comprise a heavy (VH) chain variable domain connected to a light chain variable domain (VL) on the same polypeptide chain (VH-VL) connected by a peptide linker that is too short to allow pairing between the two domains on the same chain. This forces paring with the complementary domains of another chain and promotes the assembly of a dimeric molecule with two functional antigen binding sites. In comparison to IgG, bivalent diabodies show dramatically reduced dissociation rates (Koff) as compared to the parental scFv molecules. In order to produce efficiently bispecific diabodies, the heterodimerization of two different chains needs to be preferred over the homodimerization of two equal chains. WO02/02781 describes a method in which a heterodimeric fusion protein comprising two chains where the first chain comprises one or more variable domains of immunoglobulin in a VH-VL or VL-VH format is coupled to a first heterodimerization domain and the second chain comprises one or more variable domains of immunoglobulin in a similar format as said first chain and coupled to a second heterodimerization domain interacting specifically with the first heterodimerization domain, and where at least two domains of the said first chain have intrinsic affinity to two domains of the said second chain.

Shortening of the linker between VH and VL domains to <1-2 Angstrom promotes formation of a trimeric molecule. i.e. a triabody. The triabody structure may be used as a blueprint for the design and construction of trivalent and trispecific antibody fragments (e.g. by linking the heavy and light chain V-domains of three different antibodies A, B and C to form two different chains VHA-VLB, VHB-VLC and VHC-VLA). Triabodies could bind three different or identical epitopes on the same molecule leading to very high apparent affinities especially on antigen surfaces displaying repeated epitopes (analogously to IgM). The three fold symmetry may also be of advantage in neutralizing trimeric cytokines it could mimick the three fold symmetry.

Bispecific antibodies comprising scFv molecules (U.S. Pat. No. 5,091,513) can be constructed, for instance, by genetic coupling of both scFv molecules through a polypeptide linker (U.S. Pat. No. 5,637,481). When this linker contains a heterodimerizing helix, a tetravalent Bs (scFv) 2) 2 (BiDi-body) is formed.

The C-type lectin-like domain derived binders (CTLD-derived binders) relate to binding molecules based on the family of human C-type lectins, comprising one or more C-type lectin structural units. The CTLD-derived binders all share a common structural core, serving as a scaffold holding in place the more individual loop regions, which line the ligand-binding site. Preferably, the C-type lectin-like domain derived binders (CTLD-derived binders) further comprise a second moiety consisting of a trimerisation module. Trimerisation is the process by which monomers are bound together, or ‘polymerised’, in molecular clusters of three. This additional domain binds to two other subunit domains anchored to further monomers. In this way any therapeutic protein can be formatted as a trimer, although produced as a monomer in a host cell. Polymerising proteins greatly increases their avidity, i.e. greatly increasing the availability for binding to a ligand. It has been shown that complete antibody molecules which are dimers (two monomers in complex) have 10-30-fold increases in avidity over monomer antibody fragments, while up to a 1000-fold avidity gain can be achieved by trimerisation. It appears that this increase in avidity is because only one monomer component of the trimerised molecule can bind to a specific ligand at a time but there will always be two further monomer components in very close proximity that can substitute and then substitute again ad infinitum leading to much greater and more prolonged binding to a target molecule, such as a receptor being blocked or activated.

The present invention relates in particular to Tetranectin derived CTLD binders and mannose binding protein-C (MBP-C) derived CTLD binders. The molecular architecture of tetranectin is especially suited to the development of fully human antibody analogues because it allows for simple cost-effective production and provides simple, yet sophisticated options for constructing multi- and heterovalent molecules of great versatility. Tetranectin is a 60 kDa homo-trimeric human protein assembled from three identical polypeptide chains, each comprising a coiled-coil trimerisation module and a CTLD domain. Tetranectin is found in plasma and tissue and its CTLD domains bind lysine-binding kringle-domains from Apolipoprotein(a), Hepatic Growth Factor and Plasminogen/Angiostatin. MBP-C is an important component of innate immunity system, capable of host defence against pathogens, such as bacteria, fungi, protozoa and viruses by activating the classical complement pathway independently of antibodies. MBP-C serves as well as a direct opsonin and mediates binding and uptake of pathogens by monocytes and neutrophils. The MBP-mediated complement activation is named the MBP pathway. MBP-C is a homo-oligomer composed of 32-kDa subunits. Each subunit has an N-terminal region containing cysteines involved in interchain disulfide bond formation, a collagen-like domain, a neck region, and a CTLD. Like in Tetranectin, three subunits form a structural unit, but MBP-C can oligomerise further to create higher order multimeric complexes, and an intact MBP-C cluster consists of 2-6 structural units (6-18 CTLD domains).

The trimer-derived binders of the present invention relate to a binding molecule (e.g. a monomer) comprising two moieties, and which upon expression in the host organism polymerizes to a tri-mer. The first moiety having binding activity, while the second moiety consisting of a trimerisation module. Examples of trimerisation modules include the neck region of tetranectin, made up of the 40 amino acid residue coiled-coil forming structural element, represents a versatile technology platform in rational protein engineering for trimerisation of proteins, protein domains, peptides and other compounds. The 4.5 heptaic repeats responsible for the coiled-coil formation provide a non-covalently linked trimerising element that autonomously forms the trimeric structure. The trimer has proven highly resistant to proteases and is biophysically highly stable (the trimer dissociates at an average melting temperature of around 80° C.).

Thus a further aspect of the present invention is that the monomers of the trimer-derived binders, in addition to the specific binding molecule, further comprise a trimerising domain. In the present context, the term “trimerising domain” is a peptide, a protein or part of a protein which is capable of interacting with other, similar or identical trimerising domains. The interaction is of the type that produces trimer-derived binders. Such an interaction may be caused by covalent bonds between the components of the trimerising domains as well as by hydrogen bond forces, hydrophobic forces, van der Waals forces and salt bridges. An example of a trimerising domain is disclosed in WO 95/31540 (incorporated herein by reference), which describes polypeptides comprising a collectin neck region. The amino acid sequence constituting the collectin neck region may be attached to any polypeptide of choice. Trimer-derived binders can then be made under appropriate conditions with three polypeptides comprising the collectin neck region amino acid sequence. In a further preferred embodiment, the trimerising domain is derived from tetranectin, and more specifically comprises the tetranectin trimerising structural element which is described in detail in WO 98/56906 (incorporated herein by reference).

The term “fusion protein” is used to indicate a single polypeptide or a combination of polypeptide chains where at least one polypeptide chain comprises different domains or peptide sequences derived from different sources.

According to the present invention, said binding molecule is an antibody, antibody fragment, dAb, bispecific antibody, trispecific antibody, multispecific antibody, bivalent antibody, trivalent antibody, multivalent antibody, VHH, nanobody, Fab, scFv, Fv, dAb, Fd, diabody, triabody, single chain antibody, single domain antibody, single antibody variable domain, soluble receptor, CTLD-derived binder, trimer-derived binder, ligand and/or dominant negative variant.

In an embodiment, the present invention provides a pharmaceutical composition for the use as defined herein. In a further embodiment the present invention provides a pharmaceutical composition for mucosal administration, comprising at least one binding molecule producing micro-organism as defined above.

The pharmaceutical composition according to the invention may be liquid, comprising biological active micro-organisms, or it may be solid, comprising dried micro-organisms that can be reactivated when put in a suitable environment. Micro-organisms may be dried by any system, including freeze drying and spray drying. To prepare the pharmaceutical compositions, comprising an effective amount of the micro-organism of the invention possibly combined in admixture with a pharmaceutically acceptable carrier, which can take a wide variety of forms depending on the form of preparation desired for administration. These pharmaceutical compositions are desirably in unitary dosage form suitable, preferably for oral administration. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form. In such a case, solid pharmaceutical carriers are obviously employed. In an alternative, the binding molecule producing micro-organism or the pharmaceutical composition according to the invention may be administered rectally, e.g. by an enema, i.e. the procedure of introducing liquids into the rectum and colon via the anus.

“Binding molecules producing” as used here does not imply that the micro-organism is producing the binding molecules in the pharmaceutical composition, but it means that the micro-organism is viable and can produce the binding molecules when placed in a suitable environment. Micro-organisms may be coated to facilitate the delivery into the gastro-intestinal tract. Such coating are known to the person skilled in that art and was, amongst others, described by Huyghebaert et al. (2005). The pharmaceutical composition may further comprise agents to improve the viability of the micro-organisms, such as, but not limited to trehalose. Preferably, the micro-organisms are selected from the group consisting of lactic acid bacteria and yeasts. One preferred embodiment is a pharmaceutical composition, wherein the binding molecule producing micro-organism is a Lactococcus lactis, preferably a ThyA mutant. Another preferred embodiment is a pharmaceutical composition, wherein the binding molecule producing micro-organism is a Lactobacillus sp. preferably a ThyA mutant. Preferably, said ThyA mutants are obtained by gene disruption, using the binding molecule encoding construct as insert. Still another preferred embodiment is a pharmaceutical composition wherein the binding molecule producing micro-organism is Saccharomyces cerevisiae, preferably S. cerevisiae subspecies boulardii.

Another aspect of the invention is a method of preventing, treating and/or alleviating at least one disease or disorder of the alimentary tract (gastro-intestinal tract) as defined herein, comprising administering to the alimentary tract (gastro-intestinal tract) an effective amount of a binding molecule producing micro-organism as defined herein. A preferred aspect of the invention is a method of preventing, treating and/or alleviating at least one disease or disorder of the alimentary tract (gastro-intestinal tract), comprising administering to the alimentary tract (gastro-intestinal tract) an effective amount of a binding molecule producing micro-organism capable of neutralizing the biological effect of TNFα, IL-12, IFNγ IL-23 or IL-17. Preferably said binding molecule is chosen from the group consisting of
an anti-TNFα antibody, anti-TNFα antibody fragment, anti-TNFα single antibody variable domain, soluble TNF receptor or dominant negative variant of TNFα;
anti-IL-12 antibody, anti-IL-12 antibody fragment, anti-IL-12 single antibody variable domain, soluble IL-12 receptor, dominant negative variant of IL-12 or IL-12 dAb
anti-IL-12p35 antibody, anti-IL-12p35 antibody fragment, anti-IL-12p35 single antibody variable domain, soluble IL-12p35 receptor, dominant negative variant of IL-12p35 or IL-12p35 dAb;
anti-IL-12p40 antibody, anti-IL-12p40 antibody fragment, anti-IL-12p40 single antibody variable domain, soluble IL-12p40 receptor, dominant negative variant of IL-12p40 or IL-12p40 dAb;
anti-IL-23 antibody, anti-IL-23 antibody fragment, anti-IL-23 single antibody variable domain, soluble IL-23 receptor, dominant negative variant of IL-23 or IL-23 dAb
anti-IL-23p19 antibody, anti-IL-23p19 antibody fragment, anti-IL-23p19 single antibody variable domain, soluble IL-23p19 receptor, dominant negative variant of IL-23p19 or IL-23p19 dAb
an anti-IFNγ antibody, anti-IFNγ antibody fragment, anti-IFNγ single antibody variable domain, soluble IFNγ receptor or dominant negative variant of IFNγ;
anti-IL-17 antibody, anti-IL-17 antibody fragment, anti-IL-17 single antibody variable domain, soluble IL-17 receptor, dominant negative variant of IL-17 or IL-17 dAb; and
anti-MCP-1 antibody, anti-MCP-1 antibody fragment, anti-MCP-1 single antibody variable domain, soluble IL-17 receptor, dominant negative variant of MCP-1 or MCP-1 dAb.

The way of administering can be any way known to the person skilled in the art, and includes, but is not limited to oral and rectal administration. Preferably, the way of administering is rectal or oral administration. Preferably, said disease or disorder is a disease or disorder characterized by an imbalance in TNFα production, and can be treated by TNFα inactivating compounds such as anti-TNFα antibodies, antibody fragments, single antibody variable domains, soluble receptors or dominant negative variants. Even more preferably, said disease is an inflammatory bowel disease, including but not limited to chronic colitis, ulcerative colitis and Crohn's disease. Most preferably, said disease or disorder is chronic colitis.

Preferably, said genetically modified micro-organism is a lactic acid bacterium or yeast as defined herein. In one preferred embodiment said genetically modified micro-organism is a Lactococcus lactis strain, preferably said genetically modified micro-organism is a Lactococcus lactis ThyA mutant. A specially preferred embodiment is a Lactococcus lactis ThyA mutant, wherein the gene encoding the binding molecule has been used to disrupt the THYA gene. In another preferred embodiment, said genetically modified micro-organism is a Lactobacillus sp strain, preferably said genetically modified micro-organism is a Lactobacillus ThyA mutant. A specially preferred embodiment is a Lactobacillus ThyA mutant, wherein the gene encoding the TNF-a antibody has been used to disrupt the THYA gene.

In another preferred embodiment, yeast is the binding molecule producing micro-organism. Preferably said yeast is Saccharomyces cerevisiae, even more preferably said yeast is Saccharomyces cerevisiae subsp. Boulardii.

The terms “treatment”, “treating”, and the like, as used herein include amelioration or elimination of a developed immune mediated disease or condition once it has been established or alleviation of the characteristic symptoms of such disease or condition. As used herein these terms also encompass, depending on the condition of the patient, preventing the onset of a disease or condition or of symptoms associated with a disease or condition, including reducing the severity of a disease or condition or symptoms associated therewith prior to affliction with said disease or condition. Such prevention or reduction prior to affliction refers to administration of the compound or composition of the invention to a patient that is not at the time of administration afflicted with the disease or condition. “Preventing” also encompasses preventing the recurrence or relapse-prevention of a disease or condition or of symptoms associated therewith, for instance after a period of improvement.

As used herein, the term “medicament” also encompasses the terms “drug”, “therapeutic”, “potion” or other terms which are used in the field of medicine to indicate a preparation with therapeutic or prophylactic effect.

An “effective amount” means an amount capable of lessening the spread, severity or immunocompromising effects of the diseases as indicated above. It will be apparent to those of skill in the art that the effective amount of the binding molecule producing micro-organism of this invention will depend, inter alia, upon the administration schedule, the unit dose of the binding molecule producing micro-organism administered, whether the binding molecule producing micro-organism is administered in combination with other therapeutic agents, the immune status and health of the patient, and the therapeutic activity of the particular binding molecule producing micro-organism administered. For instance, it depends on the nature and the severity of the disorder to be treated, and also on the sex, age, body weight, general health, diet, mode and time of administration, and individual responsiveness of the human or animal to be treated, on the route of administration, efficacy, metabolic stability and duration of action of the compounds used, on whether the therapy is acute or chronic or prophylactic, or on whether other active compounds are administered in addition to the agent(s) of the invention. In monotherapy for treatment of the above-indicated diseases, effective amounts per unit dose of a binding molecule producing micro-organism of the present invention range from about 0.1 μg/kg to 100 mg/kg of body weight or more, depending on the factors mentioned above. For repeated administrations over several days or longer, depending on the condition, the treatment is sustained until a desired suppression of disease symptoms occurs. A preferred dosage of the active substance of the invention may be in the range from about 1 μg/kg to about 1 mg/kg of body weight. Thus, one or more doses of about 1 μg/kg, 20 μpg/kg, 40 μg/kg or 1 mg/kg (or any combination thereof) may be administered to the patient. Such doses may be administered intermittently, e.g., every week or every three weeks 1 μg/kg to 1 mg/kg patient weight, preferably 20 μg/kg patient weight. Unit doses should be administered from twice each day to once every two weeks until a therapeutic effect is observed, preferably once every two weeks. The therapeutic effect may be measured by a variety of methods, including lymphocyte counts and clinical signs and symptoms. It will be recognized, however, that lower or higher dosages and other administration schedules may be employed.

### FIGURE LEGENDS

FIG. 1: LL-p19, LL-p40 dAb and LL-dnMCP-1 effect in vivo on acute anti-CD40 induced colitis

FIG. 2: LL-p19, LL-p40 dAb and LL-dnMCP-1 effect in vivo on established T cell-induced colitis

### EXAMPLES

### Example 1

### Material and Methods

Bacteria and Plasmids

The L. lactis strain MG1363 was used throughout this study. Bacteria were cultured in GM17 medium, i.e. M17 (Difco Laboratories, Detroit, Mich.) supplemented with 0.5% glucose. Stock suspensions of all strains were stored at −20° C. in 50% glycerol in GM17. For intragastric inoculations, stock suspensions were diluted 200-fold in fresh GM17 and incubated at 30° C. They reached a saturation density of 2×10⁹ colony-forming units (CFU) per mL within 16 hours. Bacteria were harvested by centrifugation and concentrated 10-fold in BM9 medium. (Schotte, Steidler et al. 2000). For treatment, each mouse received 100 μL of this suspension daily by intragastric catheter.

Identification of Anti-murine TNF Single Antibody Variable Domain (TNF dAb)

The generation of a cDNA of a TNF dAb was carried out in accordance with Van de Guchte et al. applied to the dAb amino acid sequence described in US2006073141. This cDNA of the TNF dAb, extended at their 3′ ends with the sequence encoding the HisG and Myc-tag, were fused to the Usp45 secretion signal (van Asseldonk, Rutten et al. 1990) downstream of the lactococcal P1 promotor (Waterfield, Le Page et al. 1995) and expressed in MG1363 (details of plasmid construction can be obtained from the authors). MG1363 strains transformed with plasmids carrying the TNF dAb coding sequence was designated LL-TNF dAb. LL-pTREX1, which is MG1363 containing the empty vector pTREX1, served as control.

Identification of Murine TNF Dominant Negative Variant (dnTNF)

The generation of a cDNA of a dnTNF was carried out in accordance with Van de Guchte et al. applied to a dnTNF sequence as derived from US2006257360. This cDNA of the dnTNF, extended at their 3′ ends with the sequence encoding the HisG and Myc-tag, were fused to the Usp45 secretion signal (van Asseldonk, Rutten et al. 1990) downstream of the lactococcal P1 promotor (Waterfield, Le Page et al. 1995) and expressed in MG1363 (details of plasmid construction can be obtained from the authors). MG1363 strains transformed with plasmids carrying the dnTNF coding sequence was designated LL-dnTNF. LL-pTREX1, which is MG1363 containing the empty vector pTREX1, served as control.

Quantification of TNF dAb and dnTNF in L. lactis Medium.

Myc-tagged LL-TNF dAb and LL-dnTNF were quantified by direct adsorption of crude L. lactis supernatants to ELISA plates (Maxisorp F96, Nunc, Rochester, N.Y.) and subsequent detection with a specific mouse mAb against the Myc epitope (Sigma, St. Louis, Mo.).

For quantification of TNF dAb and dnTNF secreted in vivo in colon tissue, the entire colon was homogenized in PBS containing 1% BSA and sonicated. The TNF dAb and dnTNF were measured in the colon supernatant with the applicable quantification protocol.

Measurement of Anti-TNF dAb and dnTNF Antibody Levels in Mouse Serum.

Mice were injected intraperitoneally with 100 μg TNF dAb or dnTNF, or intragastically with LL-TNF dAb or LL-dnTNF, daily over a 14 day-period and were subsequently bled. We coated TNF dAb and dnTNF at a concentration of 10 μg/ml in microtiterplates (NUNC Maxisorb) overnight at 4° C. The plate was washed 5 times with PBS-Tween and blocked for 2 hours at RT with PBS-1% casein. The samples were applied at a 1/50 dilution in PBS for 2 hours at RT. The plate was washed 5 times and detection was performed by incubation with rabbit-polyclonal-anti-mouse-immunoglobulin-HRP (DAKO, 3,000-fold diluted) for one hour at RT, and after washing plates were stained with ABTS/H2O2. The OD405 nm was measured.

Anti-soluble and Membrane-bound TNF Bioassay

The inhibitory effect of the TNF dAb and dnTNF on soluble mTNF (20 IU/mL) was measured in a 16 hour cytotoxicity assay using the mouse fibroblast WEHI 164 cl 13 cells in the presence of 1 μg/ml actinomycin D, as described (Espevik and Nissen-Meyer 1986). The effect of TNF dAb and dnTNF to counteract the cytotoxic effect of membrane-bound TNF was determined on the WEHI 164 cl 13 cells after adding L929 cells, expressing uncleavable, membrane-bound TNF to the cell culture (Decoster et al. 1998).

Stimulation of Macrophages with LPS

To measure the effect of TNF dAb and dnTNF on the induction of proinflammatory cytokines by LPS, MF4/4 macrophages (Desmedt et al. 1998) were incubated with TNF dAb and dnTNF (100 μg/m1). After 1 hour cells were extensively washed (3×) in a sufficient volume of PBS to completely remove all TNF dAb or dnTNF present in solution. The cells were resuspended and incubated in the presence or absence of LPS for 4 hours. The cells were washed (1×) in PBS and after 4 hours of incubation, the supernatans and cells were separated by centrifugation. To measure the soluble TNF release, the WEHI 164 cl 13 cells bioassay was used.

Animals

11-week old female BALB/c mice were obtained form Charles River Laboratories (Sulzfeld, Germany). They were housed under SPF conditions. IL-10 knockout mice (129Sv/Ev IL-10^{-/-}) (Kuhn, Lohler et al. 1993) were housed and bred under SPF conditions. The IL-10^{-/-} mice were used at 20 weeks of age, at which time chronic colitis had fully developed. All mice were fed standard laboratory feed and tap water ad libitum. The animal studies were approved by the Ethics Committee of the Department for Molecular Biomedical Research, Ghent University (File No. 04/02).

Induction of Chronic Colitis by DSS

Mice weighing approximately 21 g were induced to chronic colitis by four cycles of administration of 5% (w/v) DSS (40 kDa, Applichem, Darmstadt, Germany) in the drinking water, alternating with 10-day periods of recovery with normal drinking water. (Okayasu, Hatakeyama et al. 1990; Kojouharoff, Hans et al. 1997) Treatment was arbitrarily initiated at day 21 after the fourth cycle of DSS.

Myeloperoxidase (MPO) Assay

MPO activity in the middle colon tissue was measured as described (Bradley, Priebat et al. 1982). Pure human MPO was used as a standard (Calbiochem, San Diego, Calif.). Data are expressed as μg MPO/mm² colon tissue.

Histological Analysis

For histological analysis, the colon was removed, cleaned and opened longitudinally. A segment of 1 cm was taken from the middle part of the colon, embedded in paraffin and sectioned longitudinally. Three sections of 4 μm were cut at 200 μm intervals and stained with hematoxylin/eosin. Colon sections were numbered randomly and interpreted semiquantitatively in a blinded manner by a pathologist. The histological score is the sum of the epithelial damage and lymphoid infiltration, each ranging from 0 to 4 as described (Kojouharoff, Hans et al. 1997).

Statistical Analysis

All data are expressed as mean±SEM Parametric data were analyzed with a 1-way analysis of variance followed by a Dunnett multiple comparisons posttest. Nonparametric data (scoring) were analyzed with a Mann-Whitney test.

### EXAMPLE 1

### Example 1.1

### Anti-TNF-α dAb and dnTNF production by L. lactis In Vitro

L. lactis was transformed with the plasmids encoding TNF dAb and dnTNF. The production of the dAb and dominant negative variant was checked by Western blot and ELISA, using a strain transformed with the empty plasmid pTREX and an IL10 producing strain as reference.

### Example 1.2

### LL-TNF dAb is Bioactive and Inhibits both Soluble and Membrane Bound TNF-α

The inhibitory effect of the TNF dAb, produced by L. lactis on soluble mTNF was measured in a cytotoxicity assay using the mouse fibroblast WEHI 164 cl 13 cells as described by Espevik and Nissen-Meyer (1986). E. coli produced TNF dAb was used as a positive reference. The (purified) dAb produced by L. lactis can neutralize the soluble TNF.

The effect of dAb to counteract the cytotoxic effect of membrane-bound TNF was determined on the WEHI 164 cl 13 cells after adding L929 cells, expressing uncleavable, membrane-bound TNF to the cell culture (Decoster et al. 1998). The effect of dAb is clear with the purified form and the L. lactis produced form.

### Example 1.3

### LL-dnTNF is Bioactive and Inhibits both Soluble and Membrane Bound TNF-α

The inhibitory effect of the dnTNF, produced by L. lactis on soluble mTNF was measured in a cytotoxicity assay using the mouse fibroblast WEHI 164 cl 13 cells as described by Espevik and Nissen-Meyer (1986). E. coli produced dnTNF was used as a positive reference. The (purified) dnTNF produced by L. lactis can neutralize the soluble TNF.

The effect of dnTNF to counteract the cytotoxic effect of membrane-bound TNF was determined on the WEHI 164 cl 13 cells after adding L929 cells, expressing uncleavable, membrane-bound TNF to the cell culture (Decoster et al. 1998). The effect of dnTNF is clear with the purified form and the L. lactis produced form.

### Example 1.4

### LL-TNF dAb Effect In Vivo on Established DSS Induced Chronic Colitis

Chronic colitis was induced by DSS as described in materials and methods. Mice were daily treated with 2×10⁹ colony forming units (cfu) of either LL-pTREX1, LL-TNF dAb, or LL-mIL10. A mock treatment, and healthy mice (“watercontrol”) were used as additional control. The effect of the TNF dAb delivered by L. lactis is comparable to the protection obtained by the in situ produced IL-10.

### Example 1.5

### LL-TNF dAb Effect In Vivo on Established IL-10^{-/-} Enterocolitis

To evaluate the protection in IL-10^{-/-} enterocolitis, morbidity in 20 weeks old 129Sv/Ev IL-10^{-/-} treated and untreated mice. Each group received daily for 14 days 2×10⁹ CFU of either LL-pTREX1 (vector control), LL-TNF dAb or LL-mIL10, except the mock treated group. Both the myelperoxidase assay as well as the histological score indicate a significant protection in the LL-TNF dAb treated mice.

### Example 1.6

### Immunogenicity of TNF dAb

To evaluate a possible adverse immunogenic effect of LL-TNF dAb, mice were treated intragastically over a period of 14 days with LL-TNF dAb, using intraperitoneal injection of purified dAb as control. Anti-dAb levels were measured in the mouse serum. While interperitoneal injection of dAb is giving a clear immune response, the treatment with LL-TNF dAb is not immunogenic and proofs to be safe in that respect.

### Example 1.7

### Effect of TNF dAb on LPS Induction of Proinflammatory Cytokines

To measure the effect of TNF dAb on the induction of proinflammatory cytokines by LPS, MF4/4 macrophages (Desmedt et al. 1998) were incubated with L. lactis secreted TNF dAb. The cells were washed and then incubated with LPS. Soluble TNF release was measured using the WEHI 164 cl 13 cell toxicity assay. Pretreatment of the macrophages with L. lactis secreted TNF dAb gives a clear protection against LPS induced soluble TNF production.

### Example 1.8

### LL-dnTNF Effect In Vivo on Established DSS Induced Chronic Colitis

Chronic colitis was induced by DSS as described in materials and methods. Mice were daily treated with 2×10⁹ CFU of either LL-pTREX1, LL-dnTNF, or LL-mIL10. A mock treatment, and healthy mice (“watercontrol”) were used as additional control. The effect of the dnTNF delivered by L. lactis is comparable to the protection obtained by the in situ produced IL-10.

### Example 1.9

### LL-dnTNF Effect In Vivo on Established IL-10^{-/-} Enterocolitis

To evaluate the protection in IL-10^{-/-} enterocolitis, morbidity in 20 weeks old 129Sv/Ev IL-10^{-/-} treated and untreated mice. Each group received daily for 14 days 2×10⁹ CFU of either LL-pTREX1 (vector control), LL-dnTNF or LL-mIL10, except the mock treated group. Both the myelperoxidase assay as well as the histological score indicate a significant protection in the LL-dnTNF treated mice.

### Example 1.10

### Immunogenicity of dnTNF

To evaluate a possible adverse immunogenic effect of LL-dnTNF, mice were treated intragastically over a period of 14 days with LL-dnTNF, using intraperitoneal injection of purified dnTNF as control. Anti-dnTNF levels were measured in the mouse serum. While interperitoneal injection of dnTNF is giving a clear immune response, the treatment with LL-dnTNF is not immunogenic and proofs to be safe in that respect.

### Example 1.11

### Effect of dnTNF on LPS Induction of Proinflammatory Cytokines

To measure the effect of L. lactis secreted dnTNF on the induction of proinflammatory cytokines by LPS, MF4/4 macrophages (Desmedt et al. 1998) were incubated with dnTNF. The cells were washed and then incubated with LPS. Soluble TNF release was measured using the WEHI 164 cl 13 cell toxicity assay. Pretreatment of the macrophages with L. lactis secreted dnTNF gives a clear protection against LPS induced soluble TNF production.

### Example 2

### Materials and Methods

Bacteria and Plasmids

The L. lactis strain MG1363 was used throughout this study. Bacteria were cultured in GM17 medium, i.e. M17 (Difco Laboratories, Detroit, Mich.) supplemented with 0.5% glucose. Stock suspensions of all strains were stored at −20° C. in 50% glycerol in GM17. For intragastric inoculations, stock suspensions were diluted 200-fold in fresh GM17 and incubated at 30° C. They reached a saturation density of 2×10⁹ colony-forming units (CFU) per mL within 16 hours. Bacteria were harvested by centrifugation and concentrated 10-fold in BM9 medium (Schotte et al, 2000). For treatment, each mouse received 100 μL of this suspension daily by intragastric catheter.

Identification of Anti-murine p19 and p40 Single Antibody Variable Domain (p19 dAb and p40 dAb)

The generation of a cDNA of a p19 or p40 dAb was carried out in accordance with Van de Guchte et al. This cDNA of the p19 or p40 dAb, extended at their 3′ ends with the sequence encoding the HisG and Myc-tag, were fused to the Usp45 secretion signal (van Asseldonk et al, 1990) downstream of the lactococcal P1 promotor (Waterfield et al, 1995) and expressed in MG1363 (details of plasmid construction can be obtained from the authors). MG1363 strains transformed with plasmids carrying the p19 or p40 dAb coding sequence was designated LL-p19 dAb or p40 dAb, respectively. LL-pTREX1, which is MG1363 containing the empty vector pTREX1, served as control.

Identification of Murine MCP-1 Dominant Negative Variant (dnMCP-1)

The generation of a cDNA of a dnMCP-1 was carried out in accordance with Van de Guchte et al. applied to a dnMCP-1 sequence analogue as described in Zang et al. 1994 (Zhang et al, 1994). This cDNA of the dnMCP-1, extended at their 3′ ends with the sequence encoding the HisG and Myc-tag, were fused to the Usp45 secretion signal (van Asseldonk et al, 1990) downstream of the lactococcal P1 promotor (Waterfield et al, 1995) and expressed in MG1363 (details of plasmid construction can be obtained from the authors). MG1363 strains transformed with plasmids carrying the dnMCP-1 coding sequence was designated LL-dnMCP-1. LL-pTREX1, which is MG1363 containing the empty vector pTREX1, served as control.

Quantification of p19 or p40 dAb and dnMCP-1 in L. lactis Medium.

Myc-tagged LL-p19 or LL-p40 dAb and LL-dnMCP-1 were quantified by direct adsorption of crude L. lactis supernatants to ELISA plates (Maxisorp F96, Nunc, Rochester, N.Y.) and subsequent detection with a specific mouse mAb against the Myc epitope (Sigma, St. Louis, Mo.).

For quantification of p19 or p40 dAb and dnMCP-1 secreted in vivo in colon tissue, the entire colon was homogenized in PBS containing 1% BSA and sonicated. The p19 or p40 dAb and dnMCP-1 were measured in the colon supernatant with the applicable quantification protocol.

Measurement of Anti-p19 or Anti-p40 dAb and dnMCP-1 Antibody Levels in Mouse Serum.

Mice were injected intraperitoneally with 100 μg p19 or p40 dAb or dnMCP-1, or intragastically with LL-p19 or LL-p40 dAb or LL-dnMCP-1, daily over a 14 day-period and were subsequently bled. We coated p19 or p40 dAb and dnMCP-1 at a concentration of 10 μg/ml in microtiterplates (NUNC Maxisorb) overnight at 4° C. The plate was washed 5 times with PBS-Tween and blocked for 2 hours at RT with PBS-1% casein. The samples were applied at a 1/50 dilution in PBS for 2 hours at RT. The plate was washed 5 times and detection was performed by incubation with rabbit-polyclonal-anti-mouse-immunoglobulin-HRP (DAKO, 3,000-fold diluted) for one hour at RT, and after washing plates were stained with ABTS/H202. The OD at 405 nm was measured.

p40 dAb Bioassay

The inhibitory effect of the p40 dAb on IL-12-induced IFNγ expression was determined using freshly isolated splenocytes, which were cultured for in the presence of 10 ng/ml recombinant mIL-12 and different concentrations, ranging from 30 ng/ml to 0.003 ng/ml, of the p40 dAb. 24 h later supernatants were harvested and IFNγ expression was determined by ELISA

p19 dAb Bioassay

The inhibitory effect of the p19 dAb on IL-23-induced IL-17 expression was determined using freshly isolated splenocytes, which were cultured for in the presence of 10 ng/ml recombinant mIL-23 supplemented with 5 ng/ml PMA and different concentrations, ranging from 10 ng/ml to 0.0031 ng/ml, of the p19 dAb. 24 h or 48 h later supernatants were harvested and IL-17 expression was determined by ELISA

dnMCP-1 Bioassay

A bioassay based on MCP-1-induced phosphorylation of the mitogen-activated protein kinases (MAPK) p44 (ERK1) and p42 (ERK2) was used to test whether dnMCP-1 could block the binding of MCP-1 to its receptor. GN11 and THP-1 cells were treated with MCP-1 in 96-well plates in the presence of different concentrations of dnMCP-1. Phosphorylation was determined by using the AlphaScreen® SureFire™ Phospho-ERK 1/2 Kit (PerkinElmer). In addition, the potency of the dnMCP-1 to reduce the capacity of MCP-1 to inhibit cAMP induction in both cell lines was determined. Therefore, both cell lines were incubated with 10 μm forskolin, MCP-1 (nM concentration corresponding to IC₅₀) and different concentrations of dnMCP-1. cAMP levels were determined according to manufature's protocol (LANCE™ cAMP 384 kit, PerkinElmer).

Animals

11-week old female BALB/c mice were obtained form Charles River Laboratories (Sulzfeld, Germany). Fox Chase SCID^{-/-} mice, C.B-17 SCID (Strain code: 236, Charles River Italy) between 7-12 weeks of age. Mice were bred under specific pathogen-free conditions and kept in slim-line cages with filtered air at the VIB Department for Molecular Biomedical Research, FSVM-BL2-W248. All mice were fed standard laboratory feed and tap water ad libitum. The animal studies were approved by the Ethics Committee of the Department for Molecular Biomedical Research, Ghent University (File No. 07-032).

Induction of Innate-induced Acute Colitis by Anti-CD40

Immunodeficient mice SCID^{-/-} mice weighing approximately 21 g were injected i.p. with 250-300 μg in PBS of the anti-CD40 agonistic monoclonal antibody (eBioscience, #16-0401; clone 1C10) (Uhlig et al, 2006). Treatment was arbitrarily initiated 7 days prior to or on the same day as the anti-CD40 administration.

Induction of Chronic Colitis with Naïve CD4⁺CD45RB^{high} T Cells.

Naïve CD4⁺CD45RB^{high} T cells were isolated from the spleens of BALB/c mice using flow cytometric sorting as described (Read et al, 2000). Immunodeficient co-isogenic mice SCID^{-/-} mice weighing approximately 21 g were injected i.p. with a single dose of 4×10⁵ purified CD4⁺CD45RB^{high} T cells. Treatment was arbitrarily initiated 1 week or 4 week after transfer of T cells.

Histological Analysis

For histological analysis, the colon was removed, cleaned and opened longitudinally. A segment of 1 cm was taken from the proximal, middle and distal part of the colon, embedded in paraffin and sectioned longitudinally. Three sections of 4 μm were cut at 200 μm intervals and stained with hematoxylin/eosin. Colon sections were numbered randomly and interpreted semiquantitatively in a blinded manner by a pathologist.

Anti-CD40 innate acute colitic model: The histological score is the sum of the degree of epithelial hyperplasia, goblet cell depletion, laminia propia infiltrate and epithelial cell damage (0 no pathology, 1 mild changes, 2 intermediate, 3 severe change) as described by Uhlig et al (Uhlig et al, 2006).

T cell transfer model for chronic colitis: The histological score is the sum of the degree of epithelial hyperplasia, lymphocyte infiltration and goblet cell loss (0=normal; 1=mild epithelial hyperplasia; 2=pronounced epithelial hyperplasia with substantial inflammatory infiltrate; 3=severe hyperplasia and infiltration with marked loss of goblet cells; 4=severe hyperplasia, severe transmural inflammation, ulceration, crypt abscess and severe depletion of goblet cells) as described (Read et al, 2000). The total colonic score was obtained by adding the individual scores from the sections of proximal, mid-, and distal colon.

Statistical Analysis

All data are expressed as mean±SEM Parametric data were analyzed with a 1-way analysis of variance followed by a Dunnett multiple comparisons post-test. Nonparametric data (scoring) were analyzed with a Mann-Whitney test.

### Example 2

### Example 2.1

### Anti-p19 and Anti-p40 dAb and dnMCP-1 Production by L. lactis In Vitro

L. lactis was transformed with the plasmids encoding p19 dAb and p40 dAb and dnMCP-1. The production of the dAb and dominant negative variant was checked by Western blot and ELISA, using a strain transformed with the empty plasmid pTREX and an MO producing strain as reference.

### Example 2.2

### LL-p19 and LL-p40 dAb are Bioactive and Inhibit IFNγ and IL-17 Secretion, Respectively

The inhibitory effect of the LL-p19 dAb and LL-p40 dAb, produced by L. lactis on IL-17 and IFNγ secretion, respectively, was measured by a bioassay using freshly isolated cultured splenoytes. Recombinant mouse anti-p19 and anti-p40 were used as positive references. The (purified) p19 dAb and p40 dAB produced by L. lactis were able to inhibit the secretion of IL-17 and IFNγ by cultured splenocytes, respectively.

### Example 2.3

### dnMCP-1 is Bioactive by Inhibiting the Function of Recombinant Wild Type MCP-1

The inhibitory effect of LL-dnMCP-1 on recombinant wild type MCP-1-induced phosphorylation of the MAPK p44 and p42, and cAMP induction was tested using GN11 and THP-1 cells, which were treated with recombinant wild type MCP-1 in the presence of different concentrations of dnMCP-1. E. coli produced dnMCP-1 was used as a positive reference. The (purified) dnMCP-1 produced by L. lactis was able to inhibit phosphorylation and to reduce the capacity of MCP-1 to inhibit cAMP induction in both cell lines.

### Example 2.4

### LL-p19, LL-p40 dAb and LL-dnMCP-1 Effect In Vivo on Acute Anti-CD40 Induced Colitis

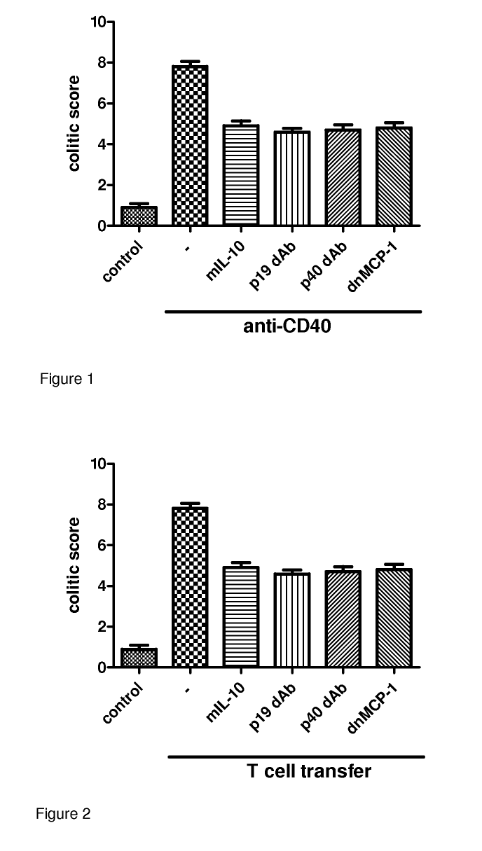

Innate acute colitis was induced by a single i.p. injection of an agonistic anti-CD40 mAb as described in materials and methods. Mice were pre-treated daily with 1×10¹⁰ colony forming units (cfu) of either LL-pTREX1, LL-p19 dAb, LL-p40 dAb, LL-dnMCP-1 or LL-mIL10. A mock treatment and healthy mice (“watercontrol”) were used as additional controls. The protective effect of the p19 dAb, p40 dAb or dnMCP-1 delivered by L. lactis is comparable to the protection obtained by the LL-mIL-10 (FIG. 1).

### Example 2.5

### LL-p19, LL-p40 dAb and LL-dnMCP-1 Effect In Vivo on Established T Cell-induced Colitis

Chronic colitis was induced by a single i.p. injection of CD4⁺CD45RB^{high} naive T cells as described in materials and methods. Mice were treated daily with 1×10¹⁰ colony forming units (cfu) of either LL-pTREX1, LL-p19 dAb, LL-p40 dAb, LL-dnMCP-1 or LL-mIL10. A mock treatment and healthy mice (“water control”) were used as additional controls. The therapeutic effect of the p19 dAb, p40 dAb or dnMCP-1 delivered by L. lactis is comparable to the therapeutic effect obtained by the in situ produced IL-10 (FIG. 2).

### Example 2.6

### Conclusion

The above experiments provide substantially the same results when repeated with binding molecules that are binding to and inhibit the biological effect of cytokines chosen from the list of IL-1β, IL-2, IL-4, IL-5, IL-6, IL-7, IL-8, IL-9, IL-12 (or its subunits IL-12p35), IL-13, IL-15, IL-16, IL-17, IL-18, IL-21, IL-23), IL-27, IL-32 (and its splice variants) and IFNα, -β and -γ.

Thus, the present invention provides for the delivery of complex, biologically active binding molecules, such as antibodies, antibody fragments, single antibody variable domains, soluble receptors, ligands and dominant negative variants, that induce an immunomodulation in a patient for the treatment of immune mediated diseases.

### REFERENCES

Bradley, et al. (1982). “Measurement of cutaneous inflammation: estimation of neutrophil content with an enzyme marker.” J Invest Dermatol 78(3): 206-9.
Espevik, T. and J. Nissen-Meyer (1986). “A highly sensitive cell line, WEHI 164 clone 13, for measuring cytotoxic factor/tumor necrosis factor from human monocytes.” J Immunol Methods 95(1): 99-105.
Huyghebaert, et al. (2005) “Development of an enteric-coated formulation containing freeze-dried, viable recombinant Lactococcus lactis for the ileal mucosal delivery of human interleukin-10.” Eur J Pharm Biopharm 60(3): 349-59
Kojouharoff, et al. (1997). “Neutralization of tumour necrosis factor (TNF) but not of IL-1 reduces inflammation in chronic dextran sulphate sodium-induced colitis in mice.” Clin Exp Immunol 107(2): 353-8.
Kuhn, R., et al. (1993). “Interleukin-10-deficient mice develop chronic enterocolitis.” Cell 75(2): 263-74.
Okayasu, et al. (1990). “A novel method in the induction of reliable experimental acute and chronic ulcerative colitis in mice.” Gastroenterology 98(3): 694-702.
Read S, et al; Malmstrom V, Powrie F (2000) Cytotoxic T lymphocyte-associated antigen 4 plays an essential role in the function of CD25(+)CD4(+) regulatory cells that control intestinal inflammation. Journal of Experimental Medicine 192: 295-302
Schotte, et al. (2000). “Secretion of biologically active murine interleukin-10 by Lactococcus lactis.” Enzyme Microb Technol 27(10): 761-765.
Uhlig et al. (2006) Differential activity of IL-12 and IL-23 in mucosal and systemic innate immune pathology. Immunity 25: 309-318
van Asseldonk, et al. (1990). “Cloning of usp45, a gene encoding a secreted protein from Lactococcus lactis subsp. lactis MG1363.” Gene 95(1): 155-60.
van de Guchte et al. (1992) “Gene expression in Lactococcus lactis” FEMS Microbiol Rev. 8:73-92
Waterfield, et al. (1995). “The isolation of lactococcal promoters and their use in investigating bacterial luciferase synthesis in Lactococcus lactis.” Gene 165(1): 9-15.
Zhang et al. (1994) “Structure/activity analysis of human monocyte chemoattractant protein-1 (MCP-1) by mutagenesis—identification of a mutated protein that inhibits MCP-1-mediated monocyte chemotaxis.” Journal of Biological Chemistry 269: 15918-15924.

1-15. (canceled)

16. A pharmaceutical composition for mucosal administration, comprising at least one binding molecule producing micro-organism.

17. The pharmaceutical composition according to claim 15, wherein said micro-organism is selected from the group consisting of lactic acid bacteria and yeasts.

18. The pharmaceutical composition according to claim 17, wherein said lactic acid bacterium is Lactococcus lactic.

19. The pharmaceutical composition according to claim 17, wherein said lactic acid bacterium is Lactobacillus sp.

20. The pharmaceutical composition according to claim 17, wherein said yeast is Saccharomyces cerevisiae.

21. The pharmaceutical composition according to claim 16, wherein said binding molecule is an antibody, antibody fragment, dAb, bispecific antibody, trispecific antibody, multispecific antibody, bivalent antibody, trivalent antibody, multivalent antibody, VHH, nanobody, Fab, scFv, Fv, dAb, Fd, diabody, triabody, single chain antibody, single domain antibody, single antibody variable domain, soluble receptor, CTLD-derived binder, trimer-derived binder, ligand and/or dominant negative variants.

22. The pharmaceutical composition according to claim 16, wherein said binding molecule has an agonizing activity.

23. The pharmaceutical composition according to claim 16, wherein said binding molecule has an antagonizing activity.

24. The pharmaceutical composition according to claim 16, wherein said binding molecules are binding to and inhibit the biological effect of cytokines selected from the list of IL-1β, IL-2, IL-4, IL-5, IL-6, IL-7, IL-8, IL-9, IL-12 (or its subunits IL-12p35 or IL-12p40), IL-13, IL-15, IL-16, IL-17, IL-18, IL-21, IL-23 (or its subunit IL-23p19), IL-27, IL-32 (and its splice variants), IFN(α, β, γ) and TNFα.

25. The pharmaceutical composition according to claim 16, wherein said binding molecules are chosen-selected from the list of:
(i) soluble cytokine receptors such as gp130,
(ii) receptor binders, such as IL-2R (CD25, CD122, CD132), EL-12 (beta1, beta2), IL-15R, IL-17R, IL-23R or IL-6R,
(iii) MIF, MIP-1α, MCP-1, RANTES and Eotaxin,
(iv) CD3/CD28, B7.1/B7.2, CD40/CD40L(CD154), HVEM, ICOS/ICOSL, OX40/X40L, CD27/CD27L(CD70), CD30/CD30L(CD153) and 41BB/41BBL;
(v) binding molecules solving the blockade of inflammation via binding to adhesion molecules from the list I-CAM1, α4 integrin and α4β7 integrin;
(vi) binding molecules having a costimulatory and agonistic effect on CD3, CTLA4 and/or PD1;
(vii) binding molecules neutralizing T-cells or B-cell activity by targeting CD25, CD20, CD52, CD95, BAFF, APRIL and/or IgE;
(viii) binding molecules solving the blockade of inflammation via binding to enzymes from the MMP family;
(ix) binding molecules asserting an anti-angiogenic effect, such as neutralizing αvβ3/α5β1 and IL-8 activity;
an anti-TNFα antibody, anti-TNFα antibody fragment, anti-TNFα single antibody variable domain, soluble TNF receptor or dominant negative variant of TNFα;
anti-IL-12 antibody, anti-IL-12 antibody fragment, anti-IL-12 single antibody variable domain, soluble IL-12 receptor, dominant negative variant of IL-12 or IL-12 dAb;
anti-IL-12p35 antibody, anti-IL-12p35 antibody fragment, anti-IL-12p35 single antibody variable domain, soluble IL-12p35 receptor, dominant negative variant of IL-12p35 or IL-12p35 dAb;
anti-IL-12p40 antibody, anti-IL-12p40 antibody fragment, anti-IL-12p40 single antibody variable domain, soluble IL-12p40 receptor, dominant negative variant of IL-12p40 or IL-12p40 dAb;
anti-IL-23 antibody, anti-IL-23 antibody fragment, anti-IL-23 single antibody variable domain, soluble IL-23 receptor, dominant negative variant of IL-23 or IL-23 dAb;
anti-IL-23p19 antibody, anti-IL-23p19 antibody fragment, anti-IL-23p19 single antibody variable domain, soluble IL-23p19 receptor, dominant negative variant of IL-23p19 or IL-23p19 dAb;
an anti-IFNγ antibody, anti-IFNγ antibody fragment, anti-IFNγ single antibody variable domain, soluble IFNγ receptor or dominant negative variant of IFNγ;
anti-IL-17 antibody, anti-IL-17 antibody fragment, anti-IL-17 single antibody variable domain, soluble IL-17 receptor, dominant negative variant of IL-17 or IL-17 dAb; and
anti-MCP-1 antibody, anti-MCP-1 antibody fragment, anti-MCP-1 single antibody variable domain, soluble IL-17 receptor, dominant negative variant of MCP-1 or MCP-1 dAb.

26. A method of preventing, treating and/or alleviating at least one disease or disorder of the gastro-intestinal tract or at least one immune mediated disease, comprising administering to the gastro-intestinal tract a micro-organism producing an effective amount of a binding molecule capable of neutralizing the biological effect of TNFα, IL-12, IFNγ, IL-23 or IL-17.

27. The method according to claim 26, wherein said binding molecule is chosen selected from the group consisting of
an anti-TNFα antibody, anti-TNFα antibody fragment, anti-TNFα single antibody variable domain, soluble TNF receptor or dominant negative variant of TNFα;
anti-IL-12 antibody, anti-IL-12 antibody fragment, anti-IL-12 single antibody variable domain, soluble IL-12 receptor, dominant negative variant of IL-12 or IL-12 dAb;
anti-IL-12p35 antibody, anti-IL-12p35 antibody fragment, anti-IL-12p35 single antibody variable domain, soluble IL-12p35 receptor, dominant negative variant of IL-12p35 or IL-12p35 dAb;
anti-IL-12p40 antibody, anti-IL-12p40 antibody fragment, anti-IL-12p40 single antibody variable domain, soluble IL-12p40 receptor, dominant negative variant of IL-12p40 or IL-12p40 dAb;
anti-IL-23 antibody, anti-IL-23 antibody fragment, anti-IL-23 single antibody variable domain, soluble IL-23 receptor, dominant negative variant of IL-23 or IL-23 dAb;
anti-IL-23p19 antibody, anti-IL-23p19 antibody fragment, anti-IL-23p19 single antibody variable domain, soluble IL-23p19 receptor, dominant negative variant of IL-23p19 or IL-23p19 dAb;
an anti-IFNγ antibody, anti-IFNγ antibody fragment, anti-IFNγ single antibody variable domain, soluble IFNγ receptor or dominant negative variant of IFNγ;
anti-IL-17 antibody, anti-IL-17 antibody fragment, anti-IL-17 single antibody variable domain, soluble IL-17 receptor, dominant negative variant of IL-17 or IL-17 dAb; and
anti-MCP-1 antibody, anti-MCP-1 antibody fragment, anti-MCP-1 single antibody variable domain, soluble IL-17 receptor, dominant negative variant of MCP-1 or MCP-1 dAb.

28. The method according to claim 26, wherein said administering is oral or rectal administering.

29. The method according to claim 26, wherein said disease or disorder is inflammatory bowel disease.

30. The method according to claim 26, wherein said micro-organism is selected from the group consisting of lactic acid bacteria and yeasts.

31. The method according to claim 30, wherein said lactic acid bacterium is Lactococcus lactis.

32. The method according to claim 30, wherein said lactic acid bacterium is Lactobacillus sp.

33. The method according to claim 30, wherein said yeast is Saccaromyces cerevisiae.

34. The method according to claim 26, wherein said binding molecule is an antibody, antibody fragment, dAb, bispecific antibody, trispecific antibody, multispecific antibody, bivalent antibody, trivalent antibody, multivalent antibody, VHH, nanobody, Fab, scFv, Fv, dAb, Fd, diabody, triabody, single chain antibody, single domain antibody, single antibody variable domain, soluble receptor, CTLD-derived binder, trimer-derived binder, ligand and/or dominant negative variants.

35. The method according to claim 26, wherein said binding molecule has an agonizing activity.

36. The method according to claim 26, wherein said binding molecule has an antagonizing activity.

37. The method according to according to claim 26, wherein said binding molecules are binding to and inhibit the biological effect of cytokines selected from the list of IL-1β, IL-2, IL-4, IL-5, IL-6, IL-7, IL-8, IL-9, IL-12 (or its subunits IL-12p35 or IL-12p40), IL-13, IL-15, IL-16, IL-17, IL-18, IL-21, IL-23 (or its subunit IL-23p19), IL-27, IL-32 (and its splice variants), IFN(α, β, γ) and TNFα.

38. The method according to claim 26, wherein said binding molecules are selected from the list of:
(i) soluble cytokine receptors such as gp130,
(ii) receptor binders, such as IL-2R (CD25, CD122, CD132), IL-12 (beta1, beta2), IL-15R, IL-17R, IL-23R or IL-6R,
(iii) MIF, MIP-1α, MCP-1, RANTES and Eotaxin,
(iv) CD3/CD28, B7.1/B7.2, CD40/CD40L(CD154), HVEM, ICOS/ICOSL, OX40/X40L, CD27/CD27L(CD70), CD30/CD30L(CD153) and 41BB/41BBL;
(v) binding molecules solving the blockade of inflammation via binding to adhesion molecules from the list I-CAM1, α4 integrin and α4β7 integrin;
(vi) binding molecules having a costimulatory and agonistic effect on CD3, CTLA4 and/or PD1;
(vii) binding molecules neutralizing T-cells or B-cell activity by targeting CD25, CD20, CD52, CD95, BAFF, APRIL and/or IgE;
(viii) binding molecules solving the blockade of inflammation via binding to enzymes from the MMP family; and
(ix) binding molecules asserting an anti-angiogenic effect, such as neutralizing αvβ3/α5β1 and IL-8 activity.

39. The method according to claim 26, wherein said immune mediated diseases are selected from the group consisting of immune mediated diseases, inflammatory diseases, autoimmune and allergic diseases and organ and bone marrow transplant rejection.

40. The method according to claim 26, wherein said immune mediated disease is a T-cell mediated disease.

41. The method according to claim 40, wherein said T-cell mediated disease is Crohn's disease.

42. The method according to claim 40, wherein said T-cell mediated disease is ulcerative colitis.
